(19) United States
(12) Patent Application Publication
(10) Pub. No.: US 2008/0249196 A1
(43) Pub. Date: Oct. 9, 2008

(54) Method for Converting Hydrocarbon-Containing Gases Into Liquids Using a Syngas with Low H2/Co Ratio

(75) Inventor: Paul Wentink, Feucherolles (FR)

Correspondence Address: AIR LIQUIDE;Intellectual Property, 2700 POST OAK BOULEVARD, SUITE 1800, HOUSTON, TX, 77056, US

(73) Assignee: L'AIR LIQUIDE Societe Anonyme pour I'Etude et I'Exploitation des Procedes Georges Claude, Paris (FR)

(21) Appl. No.: 12/088,508

(22) PCT Filed: Sep. 22, 2006
(86) PCT No.: PCT/FR06/50930

§ 371 Date: Mar. 28, 2008

(30) Foreign Application Priority Data
Sep. 28, 2005 (FR) 0552917

(51) Int. Cl. C07C 1/04

(52) U.S. Cl. 518/702

(57) ABSTRACT

The invention concerns a method for converting hydrocarbon-containing gases into hydrocarbon-containing liquids which consists in: a) producing a syngas from the hydrocarbon-containing gases, b) treating the syngas using Fischer-Tropsch process to obtain hydrocarbon-containing liquids and a residue gas comprising at least hydrogen, carbon monoxide, carbon dioxide and hydrocarbons, c) treating the residue gas using a separation process producing at least one gas stream comprising hydrogen and carbon monoxide, d) subjecting said gas stream to a reaction of carbon monoxide vapor oxidizing reaction so as to convert CO into hydrogen and CO₂, and mixing the effluent gas derived from the carbon monoxide vapor oxidizing reaction with the syngas derived form step a).

The present invention relates to a novel process for converting hydrocarbon gases into hydrocarbon liquids employing one of the processes known for generating syngas of low H₂/CO ratio followed by the Fischer-Tropsch process.

It is known to convert gaseous or solid base hydrocarbon compounds to liquid-hydrocarbon products that can be utilized in the petrochemical industry, in refineries or in the transport centre. This is because certain large deposits of natural gas are located in isolated areas remote from any region of consumption. They can then be exploited by installing what are called GTL (“gas to liquid”) conversion plants on a site close to these sources of natural gas. By converting the gases to liquid it is easier to transport to the hydrocarbons. This type of GTL conversion usually takes place by transforming the gaseous or solid base hydrocarbon compounds into a syngas comprising predominantly H₂ and CO (by partial oxidation using an oxidizing gas and/or a reaction with steam and/or CO₂), and then by treating this syngas using the Fischer-Tropsch process to obtain a product which, after condensing, results in the desired liquid hydrocarbon products. During this condensing, a waste gas is produced. This waste gas contains hydrocarbon products of low molecular weight and unreacted gases. It is generally used as fuel in one of the processes of the GTL unit, for example in a gas turbine or a combustion chamber associated with a steam turbine or in an expansion turbine associated with a compressor of the GTL unit. However, the waste gas may also be treated in order to recover these various components and to utilize them. Thus, WO 2004/092306 describes the treatment of the waste gas in order to isolate therefrom, in succession, hydrogen and then an H₂/CO mixture and CH₄, then CO₂, and then a mixture comprising hydrocarbons.

It has been observed that the step of transforming the gaseous or solid base hydrocarbon compounds to a syngas comprising predominantly H₂ and CO leads to different types of H₂/CO molar ratios depending on the nature of the reaction involved. Thus, catalytic or non-catalytic partial oxidation reactions generally result in an H₂/CO molar ratio of less than 2. Now, such H₂/CO ratio values are not always suitable for carrying out the next step of the Fischer-Tropsch process, which no longer results in high degrees of conversion of CO to liquid hydrocarbons. The unconverted CO was then burnt as fuel. In addition, the small quantity of hydrogen in the syngas may result in the formation of olefins during the Fischer-Tropsch process. These olefins disturb the implementation of the hydrocracking step. It is known to solve this hydrogen deficiency problem by adding a unit for producing hydrogen by SMR (“steam methane reforming”). However, this SMR unit requires a substantial economic investment.

The object of the present invention is to propose a novel process for converting hydrocarbon gases to hydrocarbon liquids employing a process for generating syngas that allows the H₂/CO ratio of the syngas to be increased prior to the next step of the Fischer-Tropsch process.

For this purpose, the invention relates to a process for converting hydrocarbon gases into hydrocarbon liquids, in which the following steps are carried out:

a) a syngas is produced from hydrocarbon gases, coal or residues;
b) the syngas is treated by a Fischer-Tropsch process so as to obtain hydrocarbon liquids and a waste gas comprising at least hydrogen, carbon monoxide, carbon dioxide and hydrocarbons; and
c) the waste gas is treated by a separation process for producing:
at least one gas stream comprising predominantly hydrogen,
at least one gas stream comprising hydrogen and carbon monoxide, for which the level of carbon monoxide recovery is at least 60%,
at least one gas stream comprising carbon dioxide and hydrocarbons having a carbon number of at least 2,
   characterized in that:

the gas stream comprising hydrogen and carbon monoxide for which the level of carbon monoxide recovery is at least 60% is subjected to the steam carbon monoxide oxidation reaction so as to convert CO to hydrogen and CO₂; and

the gaseous effluent resulting from the steam carbon monoxide oxidation reaction is mixed with the syngas resulting from step a) before being treated during step b).

The present invention is particularly appropriate for GTL processes in which the syngas produced in step a) has an H₂/CO ratio of at most 1.8. This is the case, for example, when the syngas is produced by catalytic or non-catalytic partial oxidation.

According to the process of the invention, this syngas is subjected to a Fischer-Tropsch reaction by bringing it into contact with a catalyst promoting this reaction. During the Fischer-Tropsch reaction, the hydrogen and CO are converted to hydrocarbon compounds of variable chain length according to the following reaction:

CO+(1+m/2n)H₂→(1/n)CₙHₘ+H₂O.

CO₂ is also produced during this reaction, for example by the following parallel reactions:

CO+H₂O→CO₂+H₂

2CO→CO₂+C.

At the exit of the reactor carrying out the Fischer-Tropsch process, the temperature of the products is generally lowered from a temperature of around 130° C. to a temperature of around 90 to 60° C. so that, on the one hand, a condensate, predominantly composed of water and hydrocarbon liquids having a carbon number greater than 4 is obtained and, on the other hand, a waste gas comprising at least hydrogen, carbon monoxide, hydrocarbons having a carbon number of at most 6, carbon dioxide and generally also nitrogen is obtained.

According to the process of the invention, this waste gas is subjected to a separation process that produces:

at least one gas stream comprising predominantly hydrogen;

at least one gas stream comprising hydrogen and carbon monoxide for which the level of carbon monoxide recovery is at least 60%; and

at least one gas stream comprising carbon dioxide and hydrocarbons having a carbon number of 2.

According to the invention, the level of recovery of a compound in one of the gas streams resulting from the separation process corresponds to the volume or molar quantity of said compound present in the waste gas that is separated from the waste gas and which is produced in said gas stream resulting from the separation process relative to the total volume or molar quantity of this compound present in the waste gas. In the case of the gas stream whose level of hydrogen and carbon monoxide recovery is at least 60%, the 60% recovery in condition applies to the CO compound relative to the quantity of CO initially present in the waster gas. According to the invention, the expression “gas stream comprising predominantly a compound” is understood to mean a gas stream of which the concentration in this compound is greater than 50% by volume. According to the invention, the separation process of treating the waste gas is advantageously a PSA (“pressure swing adsorption”) separation process. This PSA separation process is carried out using a PSA separation unit for obtaining at least the three aforementioned gas streams.

The gas stream comprising predominantly hydrogen generally has a hydrogen concentration of greater than 98% by volume. On account of its purity, this stream is used in a unit for hydrocracking the liquid hydrocarbons produced by the Fischer-Tropsch process.

In general, in respect of the second gas stream, based on H₂ and CO, the level of carbon monoxide recovery is higher than the level of hydrogen recovery. The level of recovery is about 60 to 75% in the case of carbon monoxide and about 15 to 85% in the case of hydrogen, the level of hydrogen recovery in this second stream being dependent on the level of hydrogen recovery in the first stream. This second stream generally also includes methane—about 50% of the methane initially present in the waste gas is present in the second stream based on H₂ and CO. Finally, this second stream also includes nitrogen.

The third and final stream is a complementary stream comprising CO₂ and the hydrocarbons initially present in the waste gas. This stream also includes the rest of the CH₄ initially present in the waste gas and also nitrogen, hydrogen and CO.

Preferably, each adsorber of the PSA separation unit is made up of at least two adsorbent beds,

the first bed being made up of a mixture of silica gel, activated charcoal and either carbon-containing molecular sieves or zeolites, having pore sizes between 3.4 and 5 Å and preferably between 3.7 and 4.4 Å, or titanosilicates with a mean pore size of between 3.4 and 5 Å, and preferably between 3.7 and 4.4 Å,

the second bed being made up of an alumina-rich zeolite.

The order of the two adsorbent beds is the following, depending on the direction of flow of the waste gas in the absorber: the first bed then the second bed.

Depending on the various pressure cycles, the PSA separation process makes it possible to obtain, in succession:

the gas stream under high pressure comprising predominantly hydrogen;

the gas stream under high pressure for which the level of carbon monoxide recovery is at least 60%; and then

the complementary gas stream comprising predominantly carbon dioxide and hydrocarbons having a carbon number of at least 2.

The silica gel makes it possible to adsorb the hydrocarbon compounds and especially the hydrocarbon compounds having a carbon number of at least 3. Preferably, the silica gel used has an alumina (Al₂O₃) concentration of less than 1% by weight. However, the silica gel lets through the H₂ and CO. The zeolite or the carbon-containing molecular sieves with a mean pore size of between 3.4 and 5 Å, and preferably between 3.7 and 4.4 Å, make it possible to adsorb CO2 and, at least partially, CH₄. The activated charcoal makes it possible to adsorb oxygen-containing hydrocarbons, such as alcohol, aldehydes, esters, etc. The alumina-rich zeolite stops the CO and N2 components.

According to one of the essential features of the invention, the gas stream for which the level of carbon monoxide recovery is at least 60% is heated and mixed with steam before being subjected to the steam carbon monoxide oxidation reaction. The CO-rich gas is heated by bringing it into contact with the products leaving the reactor and is mixed with steam at a temperature of about 320° C. in the presence of an iron-based catalyst. Since the reaction is exothermic, the heat of the CO2 produced by the oxidation reaction may be extracted by bringing it into contact with the reactive gas based on H₂ and CO before its introduction into the Fischer-Tropsch reactor. The (steam/gas stream comprising H₂ and CO) molar ratio is about 1.5 to 2. For certain Fischer-Tropsch processes sensitive to steam, the gaseous product resulting from the CO oxidation reaction is cooled to a temperature allowing the water to be removed therefrom, and then this effluent is reheated before being introduced into the Fischer-Tropsch reactor.

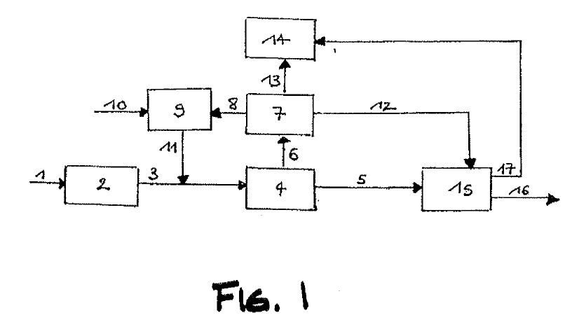

FIG. 1 illustrates the process according to the invention. Natural gas is introduced into a syngas production unit 2 forming a syngas 3, which is treated in a Fischer-Tropsch unit 4 for producing hydrocarbon liquids 5. These liquids may be hydrocracked in a hydrocracking unit 15 in order to produce shorter chain-length hydrocarbon liquids 16. The Fischer-Tropsch unit 4 also produces a waste gas 6 that is treated by the unit 7, preferably a PSA unit, resulting in:

a hydrogen-rich gas stream 12 that is used in the hydrocracking unit 15,

a gas stream 13 comprising carbon dioxide and hydrocarbons having a carbon number of at least 2, which is burned in a boiler 14; and

a gas stream 8 comprising hydrogen and carbon monoxide for which the level of carbon monoxide recovery is at least 60%.

The gas stream undergoes a carbon monoxide oxidation reaction by reaction with steam 10 in the unit 9. The gaseous effluent 11 from this reaction is mixed with the syngas 3 before its treatment by the Fischer-Tropsch unit 4.

By carrying out the process described above, it therefore becomes possible to reduce the hydrogen production operating costs since the carbon monoxide steam oxidation process uses a gas which normally would simply be used as fuel. Thus, a 12% reduction in natural gas consumption may be achieved. Furthermore, the cost of investing in an SMIR unit is avoided.

1-6. (canceled)

7. A process for converting hydrocarbon gases into hydrocarbon liquids, in which the following steps are carried out:
a) a syngas is produced from hydrocarbon gases, coal or residues;
b) the syngas is treated by the Fischer-Tropsch process so as to obtain hydrocarbon liquids and a waste gas comprising at least hydrogen, carbon monoxide, carbon dioxide and hydrocarbons; and
c) the waste gas is treated by a separation process for producing:
1) at least one gas stream comprising predominantly hydrogen;
2) at least one gas stream comprising hydrogen and carbon monoxide, for which the level of carbon monoxide recovery is at least 60%; and
3) at least one gas stream comprising carbon dioxide and hydrocarbons having a carbon number of at least 2,
wherein:
the gas stream comprising hydrogen and carbon monoxide for which the level of carbon monoxide recovery is at least 60% is subjected to a steam carbon monoxide oxidation reaction so as to convert CO to hydrogen and CO₂; and
the gaseous effluent resulting from the steam carbon monoxide oxidation reaction is mixed with the syngas resulting from step a) before being treated during step b).

8. The process of claim 7, wherein the syngas produced in step a) has an H₂/CO ratio of at most 1.8.

9. The process of claim 7, wherein the syngas is produced during step a) by catalytic or non-catalytic partial oxidation.

10. The process of claim 7, wherein the process of treating the waste gas during step b) employs a PSA separation unit.

11. The process of claim 10, wherein each adsorber of the PSA separation unit is made up of at least two adsorbent beds,
a) the first bed being made up of a mixture of silica gel, activated charcoal and either carbon-containing molecular sieves or zeolites, having pore sizes between 3.4 and 5 Å and preferably between 3.7 and 4.4 Å, or a titanosilicate with a mean pore size of between 3.4 and 5 Å, and preferably between 3.7 and 4.4 Å, and
b) the second bed being made up of an alumina-rich zeolite.

12. The process of claim 7, wherein the gas stream for which the level of carbon monoxide recovery is at least 60% is heated and mixed with steam before being subjected to the steam carbon monoxide oxidation reaction.
